(19) United States
(12) Patent Application Publication
(10) Pub. No.: US 2026/0297121 A1
(43) Pub. Date: Oct. 1, 2026

(54) MITHRAMYCIN (MTM) 2’ OXIMES

(71) Applicant: University of Kentucky Research Foundation, Lexington, KY (US)

(72) Inventors: Jon S. Thorson, Lexington, KY (US); Markos Leggas, Lexington, KY (US); Yang Liu, Lexington, KY (US); Aarajana Shrestha, Lexington, KY (US)

(21) Appl. No.: 19/474,614

(22) PCT Filed: Apr. 11, 2024
(86) PCT No.: PCT/US2024/024117

§ 371 Date: Oct. 10, 2025

Related U.S. Application Data
(60) Provisional application No. 63/495,407, filed on Apr. 11, 2023.

(51) Int. Cl. C07H 15/26; A61K 31/7056; A61K 31/706; A61K 31/7064; A61K 31/7072; A61P 35/00

(52) U.S. Cl. CPC C07H 15/26; A61K 31/7056; A61K 31/706; A61K 31/7064; A61K 31/7072; A61P 35/00

(57) ABSTRACT

Compounds described herein are mithramycin (MTM) oxide (OX) derivatives. These compounds are useful for treatment of cancers and neuro-diseases.

### RELATED APPLICATIONS

This application claims priority from U.S. Provisional Application Ser. No. 63/495,407 filed Apr. 11, 2023, the entire disclosure of which is incorporated herein by this reference.

### GOVERNMENT INTEREST

This invention was made with government support under grant number P20 GM130456 and R37 AI052218 awarded by the National Institutes of Health. The government has certain rights in the invention.

### REFERENCE TO AN ELECTRONIC SEQUENCE LISTING

The contents of the electronic sequence listing (Thorson UKRF 2815.xml; Size: 5532 bytes; and Date of Creation: Apr. 9, 2024) is herein incorporated by reference in its entirety.

### TECHNICAL FIELD

The present disclosure relates to compounds that are mithramycin (MTM) Oxime (OX) derivatives and their use in the treatment of cancers and neuro-diseases.

### INTRODUCTION

All members of the erythroblast transformation-specific (ETS) transcription factor-family contain an Ets-domain, which consists of approximately 80 amino acids with four tryptophan repeats. The Ets-domain binds to double-stranded DNA of target genes containing a GGAA/T core motif and different flanking regions. Exemplary ETS transcription factors include friend leukemia integration 1 transcription factor (FLI1), v-ets avian erythroblastosis virus E26 oncogene-like transcription factor (ERG), and SPI1 or PU.1 transcription factor (SPI1).

ETS transcription factors can impact the expression of genes that are involved in various processes, such as cellular proliferation, differentiation, development, transformation, and apoptosis, and can have implications in connection with cancer. For example, FLI1 aberrant regulation is often associated with malignant transformation and is associated with chromosomal abnormalities in humans. In Ewing Sarcoma and primitive neuroectodermal tumors, for example, a chromosomal translocation results in a chimeric EWS-FLI1 fusion protein, containing the 5′ region of EWS (Ewing sarcoma breakpoint region 1) and the 3′ ETS region of Fli-1 (Delattre et al., Nature. 1992 Sep. 10; 359 (6391): 162-5). This oncoprotein acts as an aberrant transcriptional activator with strong transforming capabilities.

FLI1 and homologous transcription factors also have been implicated in human leukemias, such as Acute Myelogenous Leukemia (AML), involving loss or fusion of the tel gene, as well as other malignancies including clear-cell sarcoma, myxoid liposarcoma, desmoplastic small round cell tumor, myxoid chondrosarcoma, acute myeloid leukemia, congenital fibrosarcoma, prostate cancer and pancreatic cancer.

Another ETS transcription factor, ERG, is implicated in several cancers. Aberrant ERG regulation has been shown to be associated with diseases including Ewing sarcoma, acute myeloid leukemia (AML), prostate cancer, acute lymphoblatic leukemia (ALL), Alzheimer's disease (AD), and Down syndrome (DS).

Although ETS transcription factors such as FLI1, ERG, and SPI1 have been identified as critical targets in diseases such as cancer, no therapies have yet moved from bench to bedside that could impact the outcome of this disease. Ewing sarcoma, which affects primarily children and young adults is a difficult cancer to treat. Current therapy with a combination of severely cytotoxic drugs provides up to 60% long-term survival, but the cancer often recurs.

Mithramycin or mithramycin A (MTM) is an aureolic acid-type polyketide drug produced by various soil bacteria of the genus Streptomyces and was found to possess activity against a wide variety of human cancers.¹⁻²

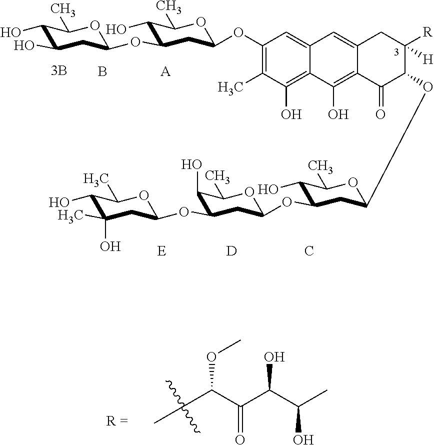

MTM was clinically evaluated in the 1960s and 70s as an agent for the chemotherapy of various cancers. As noted above, despite some remarkable success using MTM as a single agent, the results were mixed due to its narrow therapeutic index and considerable variation in patients' ability to tolerate the drug.³ Another concern was the lack of understanding of MTM's mode-of-action. Taken together these limitations limited clinical use of MTM as a chemotherapeutic agent and it has now been largely abandoned.⁴ Interest in MTM was renewed recently, after the drug was identified as the top inhibitor of the ETS transcription factor fusion, EWS-FLI1, in a screen of more than 50,000 natural products and synthetic compounds. FLI1 and ERG are ETS transcription factors that are expressed as fusions with EWS and are the primary cause of Ewing sarcoma.⁵⁻⁶

Aside from Ewing sarcoma, aberrant ETS transcription factors contribute significantly to the malignancy of prostate cancer, leukemia and lymphoma. With respect to prostate cancer, approximately 50% of patients express a truncated form of ERG as a result of the TMPRSS2 (transmembrane protease, serine 2)-ERG gene fusion.⁷ Interestingly, the DNA binding domain of ERG and FLI1 is conserved and thus molecules that interfere with the activity of one should also inhibit the other. Given the importance of these aberrant transcription factors in driving malignancy, the clinical use of MTM gave investigators hope for a “targeted” therapy. This was tested in a recent national cancer institute (NCI) conducted clinical study where Ewing sarcoma patients were enrolled to assess the utility of MTM in a population of patients, all of whom express ETS fusions. Unfortunately, the results were inconclusive because the trial was terminated early, due to toxicities.

Therefore, MTM analogues that are less toxic and more selective against cancers cells are needed. MTM has high potential in the fight against cancer and new and improved analogues would find clinical relevance. A need thus exists to improve the performance, selectivity, and efficacy of MTM.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes a compound having the following formula:

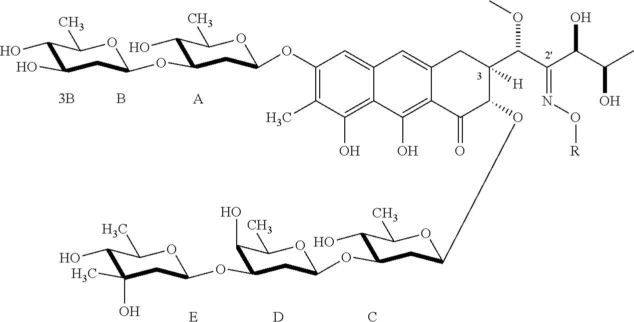

or a pharmaceutically acceptable salt thereof.

In some embodiments of the compound, wherein R is

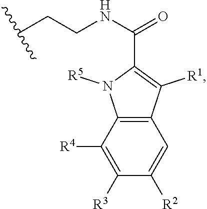

in which R¹ is H, halogen, phenyl, halogen-substituted phenyl, alkyl-substituted pyrazole, thiophene, isoxazole, alkyl-substituted isoxazole, or cyclopropane; R² is OH, OCH₃, CF₃, halogen, or alkyl-substituted pyrazole; R³ is H or OCH₃; R⁴ is H or halogen, and R⁵ is H or alkyl.

In some embodiments of the compound, R is

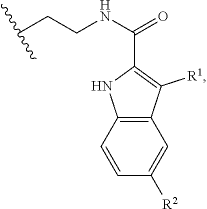

in which R¹ is H, halogen, phenyl, halogen-substituted phenyl, alkyl-substituted pyrazole, thiophene, isoxazole, alkyl-substituted isoxazole, or cyclopropane; and R² is OH, OCH₃, CF₃, halogen, or alkyl-substituted pyrazole. In some embodiments, R¹ is H, halogen, phenyl, halogen-substituted phenyl, alkyl-substituted pyrazole, thiophene, isoxazole, alkyl-substituted isoxazole, or cyclopropane; and R² is CF₃ or halogen.

In some embodiments of the compound, R is selected from the groups as set forth in Table 1, provided herein.

In some embodiments, R comprises an acyl tryptophan or derivative thereof. In some embodiments, the group comprises amino benzo[d]thiazol, N,N-diethyl-amine, azetidine, 3,3-dimethylazetidine, or morpholine, or derivative thereof. In some embodiments, R comprises alkyl benzo[d]thiazole or alkyl carboxamide benzo[d]thiazole. In some embodiments, R comprises alkyl carboxamide 4-isoquinoline, alkyl carboxamide 5,7-dimethyl-[1,2,4]triazolo[4,3-a]pyrimidine, alkyl carboxamide 1,3-dimethyl-2,4-dioxo-2,3,4,7-tetrahydro-1H-pyrrolo[2,3-d]pyrimidine.

In some embodiments, R comprises an alkyl indole or alkyl carboxamide indole. In some embodiments, the indole is a substituted indole group. The group comprises 5-hydroxy-indole, 5-methoxy-indole, 5-fluoro-indole, 5-chloro-indole, 5-bromo-indole, 5-trifluoromethyl-indole, 5-trifluoromethoxy-indole, 5-(1-methyl-1H-pyrazol-4-yl)-indole. In some embodiments, R comprises a 5,6-dimethoxy-indol, 5,7-dichloro-indole. In some embodiments, R comprises a (5-chloro-3-cyclopropyl)-indole, 5-chloro-3-(isoxazole-4-yl)-indole, 5-chloro-(3,5-dimethylisoxazole-4-yl)-indole, 5-chloro-3-(1-methyl-1H-pyrazol-4-yl)-indole, 5-chloro-3-(1-methyl-1H-pyrazol-5-yl)-indole, 5-chloro-3-(pyrimidin-5-yl)-indole, 5-chloro-3-(3-chloro-4-fluorophenyl)-indole, 5-chloro-3-(thiophen-2-yl)-indole, 5-chloro-3-phenyl-indole, 5-chloro-3-(4-chlorophenyl)-indole, 5-chloro-3-(4-chlorophenyl)-1-methyl-indole, 5-chloro-3-(4-chlorophenyl)-1-isobutyl-indole, 3-(4-chlorophenyl)-5-hydroxy-indole, 3-(4-chlorophenyl)-5-methoxy-indole, 3-(4-chlorophenyl)-5-fluoro-indole, 3-(4-chlorophenyl)-5-(trifluoromethyl)-indole, 3-phenyl-5-(trifluoromethyl)-indole or 3-(3-chloro-4-fluorophenyl)-5-(trifluoromethyl)-indole. In some embodiments, the group comprises(S)—N-(1-(1H-indol-3-yl) butan-2-yl)benzo[d]thiazole-5-carboxamide, (S)—N-(1-(1H-indol-3-yl) butan-2-yl)-5-chloro-1H-indole-2-carboxamide, (R)—N-(1-(1H-indol-3-yl) butan-2-yl)benzo[d]thiazole-5-carboxamide, or (R)—N-(1-(1H-indol-3-yl) butan-2-yl)-5-chloro-1H-indole-2-carboxamide.

The compounds of the presently-disclosed subject matter can be used for the treatment of cancer, such as brain, colon, prostate, lung, breast, esophageal, pancreatic, skin, Ewing sarcoma, any type of blood cancer etc. MTM derivatives are also neuroprotective and can be used to treat various neuro-diseases, such as Huntington disease, etc.

The presently-disclosed subject matter further includes composition comprising the compound, as disclosed herein, and a pharmaceutically-acceptable carrier.

The presently-disclosed subject matter further includes a method of treating cancer or neuro-disease in a patient in need thereof, which involves administering to the patient a therapeutically effective amount of a compound, as disclosed herein. In some embodiments, the method comprises treating Ewing sarcoma, prostate cancer, colon cancer, lung cancer, leukemia, lymphoma, rhabdomyosarcoma or rhabdoid tumor.

The presently-disclosed subject matter further includes a method for selectively modulating the activity of a target ETS transcription factor in a patient in need thereof, which involves administering to the patient a therapeutically effective amount of a compound, as disclosed herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A and 1B. Pharmacokinetics in mice following bolus IV dosing. YL-C09 analogue compared to mithramycin (MTM) dosed separately (FIG. 1A), and YL-B171, YL-C110 and MTM dosed in cassette format (FIG. 1B).

FIG. 2A-2E. Cytotoxicity plots and estimates of GI₅₀ and AUC in rhabdomyosarcoma cells of embryonal (JR1 (FIG. 2A), RD (FIG. 2B)) and alveolar (RH30 (FIG. 2C), RH41 (FIG. 2D)) histologies, and a fibroblast cell line (HS68 (FIG. 2E)).

FIG. 3A-3D. Western blot (FIGS. 3A and 3C) and semiquantitative assessment of drug effects (FIGS. 3B and 3D).

### BRIEF DESCRIPTION OF THE SEQUENCE LISTING

SEQ ID NO: 1 is an amino acid sequence for a DNA-binding domain (DBD) of target ETS transcription factor.

SEQ ID NO: 2 is another amino acid sequence for a DNA-binding domain (DBD) of target ETS transcription factor.

SEQ ID NO: 3 is an amino acid sequence of FLI1 transcription factor.

SEQ ID NO: 4 is an amino acid sequence of ERG transcription factor.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes MTM-OX derivatives useful for treatment of cancer and other conditions, including diseases associated with an aberrant erythroblast transformation-specific transcription factor.

Compounds as disclosed herein have the structure of Formula I

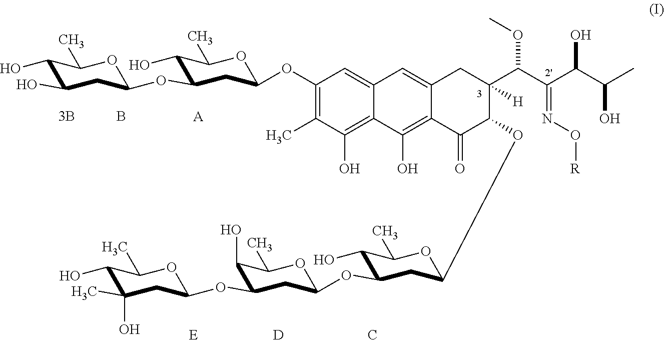

In some embodiments of the present disclosure, the compound has the structure of Formula I, wherein R is selected from the groups as set forth in Table 1.

TABLE 1

| | R of Formula (I) | | R of Formula (I) |
|---|---|---|---|
| YL-B152 | 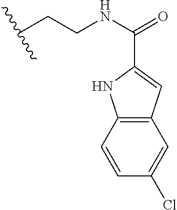 [SMILES: *CCNC(=O)c1cc2cc(Cl)ccc2[nH]1] | YL-B158 | 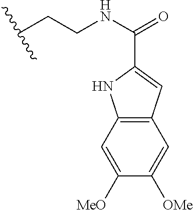 [SMILES: *CCNC(=O)c1cc2cc(OC)c(OC)cc2[nH]1] |
| YL-B160 | 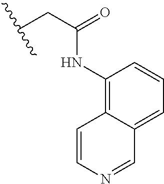 [SMILES: *CC(=O)Nc1cccc2cnccc12] | YL-B162 | 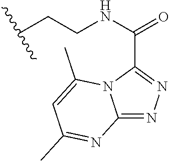 [SMILES: *CCNC(=O)c1nnc2nc(C)cc(C)n12] |
| YL-B166 | 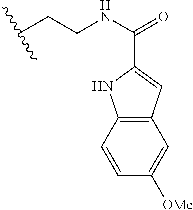 [SMILES: *CCNC(=O)c1cc2cc(OC)ccc2[nH]1] | YL-B167 | 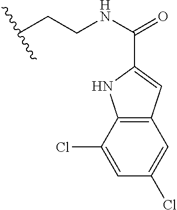 [SMILES: *CCNC(=O)c1cc2cc(Cl)cc(Cl)c2[nH]1] |
| YL-B168 | 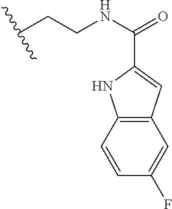 [SMILES: *CCNC(=O)c1cc2cc(F)ccc2[nH]1] | YL-B169 | 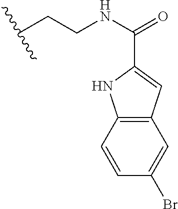 [SMILES: *CCNC(=O)c1cc2cc(Br)ccc2[nH]1] |
| YL-B170 | 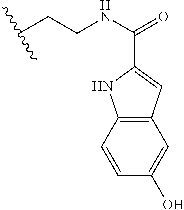 [SMILES: *CCNC(=O)c1cc2cc(O)ccc2[nH]1] | YL-B171 | 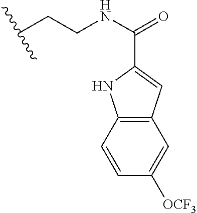 [SMILES: *CCNC(=O)c1cc2cc(OC(F)(F)F)ccc2[nH]1] |
| YL-B175 | 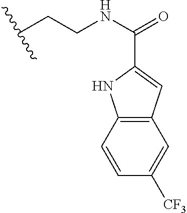 [SMILES: *CCNC(=O)c1cc2cc(C(F)(F)F)ccc2[nH]1] | | |
| YL-B176 | 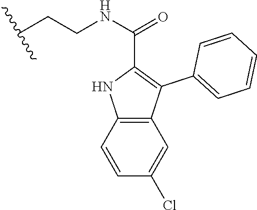 [SMILES: *CCNC(=O)c1[nH]c2ccc(Cl)cc2c1-c1ccccc1] | YL-B179 | 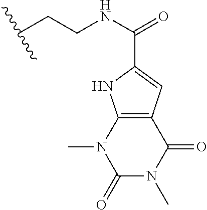 [SMILES: *CCNC(=O)c1cc2c(=O)n(C)c(=O)n(C)c2[nH]1] |
| YL-C9 | 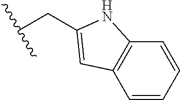 [SMILES: *Cc1cc2ccccc2[nH]1] | YL-C20 | 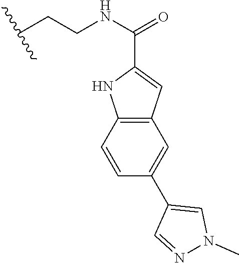 [SMILES: *CCNC(=O)c1cc2cc(-c3cnn(C)c3)ccc2[nH]1] |
| YL-C39 | 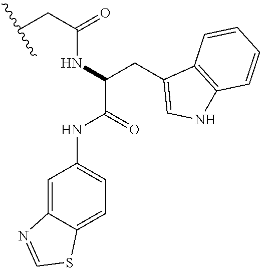 [SMILES: *CC(=O)N[C@@H](Cc1c[nH]c2ccccc12)C(=O)Nc1ccc2scnc2c1] | YL-C51 | 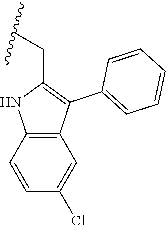 [SMILES: *Cc1[nH]c2ccc(Cl)cc2c1-c1ccccc1] |
| YL-C52 | 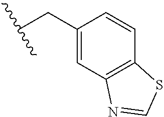 [SMILES: *Cc1ccc2scnc2c1] | YL-C53 | 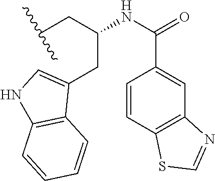 [SMILES: *C[C@@H](Cc1c[nH]c2ccccc12)NC(=O)c1ccc2scnc2c1] |
| YL-C54 | 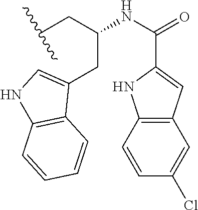 [SMILES: *C[C@@H](Cc1c[nH]c2ccccc12)NC(=O)c1cc2cc(Cl)ccc2[nH]1] | YL-C55 | 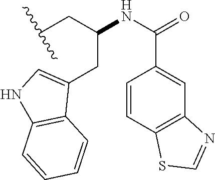 |
| YL-C56 | 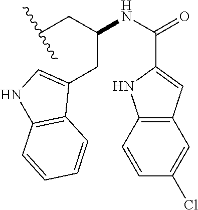 | YL-C61 | 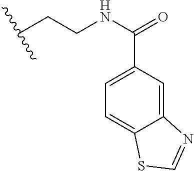 [SMILES: *CCNC(=O)c1ccc2scnc2c1] |
| YL-C66 | 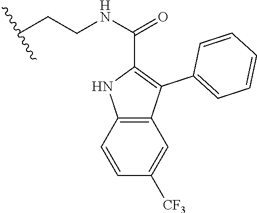 [SMILES: *CCNC(=O)c1[nH]c2ccc(C(F)(F)F)cc2c1-c1ccccc1] | YL-C67 | 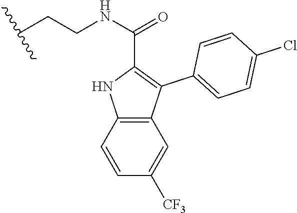 [SMILES: *CCNC(=O)c1[nH]c2ccc(C(F)(F)F)cc2c1-c1ccc(Cl)cc1] |
| YL-C68 | 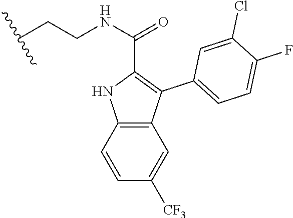 [SMILES: *CCNC(=O)c1[nH]c2ccc(C(F)(F)F)cc2c1-c1ccc(F)c(Cl)c1] | YL-C82 | 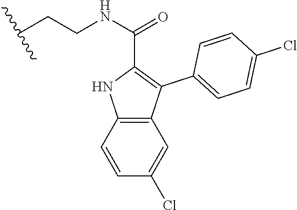 [SMILES: *CCNC(=O)c1[nH]c2ccc(Cl)cc2c1-c1ccc(Cl)cc1] |
| YL-C95 | 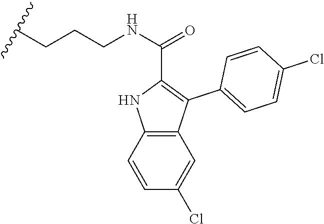 [SMILES: *CCCNC(=O)c1[nH]c2ccc(Cl)cc2c1-c1ccc(Cl)cc1] | YL-C98 | 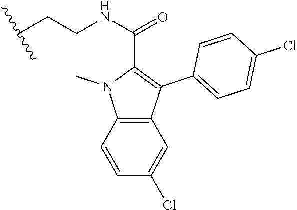 [SMILES: *CCNC(=O)c1c(-c2ccc(Cl)cc2)c2cc(Cl)ccc2n1C] |
| YL-C100 | 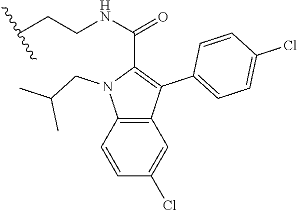 [SMILES: *CCNC(=O)c1c(-c2ccc(Cl)cc2)c2cc(Cl)ccc2n1CC(C)C] | YL-C102 | 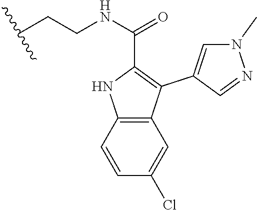 [SMILES: *CCNC(=O)c1[nH]c2ccc(Cl)cc2c1-c1cnn(C)c1] |
| YL-C104 | 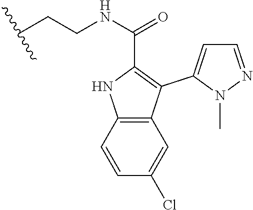 [SMILES: *CCNC(=O)c1[nH]c2ccc(Cl)cc2c1-c1ccnn1C] | YL-C106 | 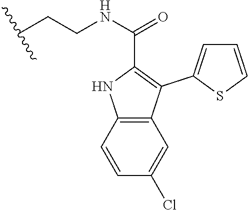 [SMILES: *CCNC(=O)c1[nH]c2ccc(Cl)cc2c1-c1cccs1] |
| YL-C108 | 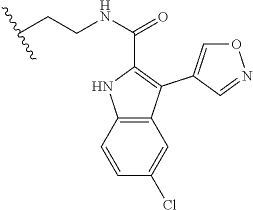 [SMILES: *CCNC(=O)c1[nH]c2ccc(Cl)cc2c1-c1cnoc1] | YL-C110 | 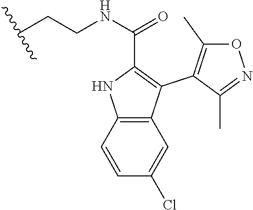 [SMILES: *CCNC(=O)c1[nH]c2ccc(Cl)cc2c1-c1c(C)noc1C] |
| YL-C114 | 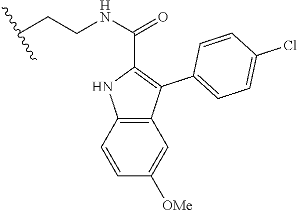 [SMILES: *CCNC(=O)c1[nH]c2ccc(OC)cc2c1-c1ccc(Cl)cc1] | YL-C116 | 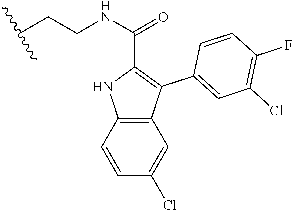 [SMILES: *CCNC(=O)c1[nH]c2ccc(Cl)cc2c1-c1ccc(F)c(Cl)c1] |
| YL-C118 | 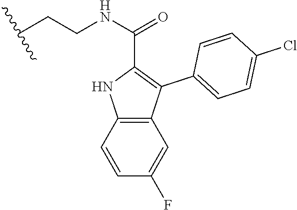 [SMILES: *CCNC(=O)c1[nH]c2ccc(F)cc2c1-c1ccc(Cl)cc1] | YL-C120 | 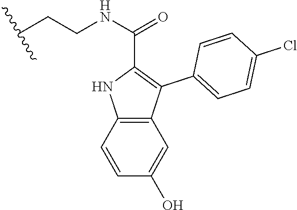 [SMILES: *CCNC(=O)c1[nH]c2ccc(O)cc2c1-c1ccc(Cl)cc1] |
| YL-C122 | 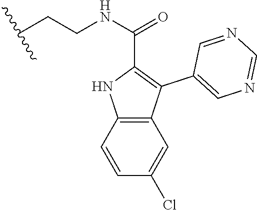 [SMILES: *CCNC(=O)c1[nH]c2ccc(Cl)cc2c1-c1cncnc1] | YL-C124 | 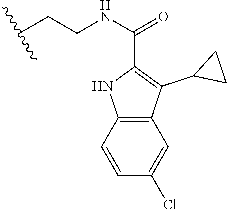 [SMILES: *CCNC(=O)c1[nH]c2ccc(Cl)cc2c1C1CC1] |

In some embodiments, the compound has the structure of Formula I, wherein R is

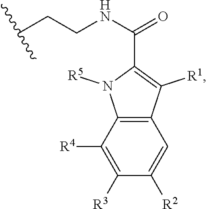

in which R¹ is H, halogen, phenyl, halogen-substituted phenyl, alkyl-substituted pyrazole, thiophene, isoxazole, alkyl-substituted isoxazole, or cyclopropane; R² is OH, OCH₃, CF₃, halogen, or alkyl-substituted pyrazole; R³ is H or OCH₃; R⁴ is H or halogen, and R⁵ is H or alkyl.

In some embodiments, the compound has the structure of Formula I, wherein R is

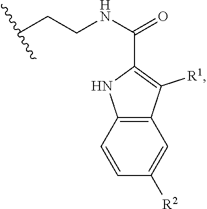

in which R¹ is H, halogen, phenyl, halogen-substituted phenyl, alkyl-substituted pyrazole, thiophene, isoxazole, alkyl-substituted isoxazole, or cyclopropane; and R² is OH, OCH₃, CF₃, halogen, or alkyl-substituted pyrazole. In some embodiments, R¹ is H, halogen, phenyl, halogen-substituted phenyl, alkyl-substituted pyrazole, thiophene, isoxazole, alkyl-substituted isoxazole, or cyclopropane; and R² is CF₃ or halogen.

In some embodiments, R comprises an acyl tryptophan or derivative thereof. In some embodiments, the group comprises amino benzo[d]thiazol, N,N-diethyl-amine, azetidine, 3,3-dimethylazetidine, or morpholine, or derivative thereof. In some embodiments, R comprises alkyl benzo[d]thiazole or alkyl carboxamide benzo[d]thiazole. In some embodiments, R comprises alkyl carboxamide 4-isoquinoline, alkyl carboxamide 5,7-dimethyl-[1,2,4]triazolo[4,3-a]pyrimidine, alkyl carboxamide 1,3-dimethyl-2,4-dioxo-2,3,4,7-tetrahydro-1H-pyrrolo[2,3-d]pyrimidine.

In some embodiments, R comprises an alkyl indole or alkyl carboxamide indole. In some embodiments, the indole is a substituted indole group. The group comprises 5-hydroxy-indole, 5-methoxy-indole, 5-fluoro-indole, 5-chloro-indole, 5-bromo-indole, 5-trifluoromethyl-indole, 5-trifluoromethoxy-indole, 5-(1-methyl-1H-pyrazol-4-yl)-indole. In some embodiments, R comprises a 5,6-dimethoxy-indol, 5,7-dichloro-indole. In some embodiments, R comprises a (5-chloro-3-cyclopropyl)-indole, 5-chloro-3-(isoxazole-4-yl)-indole, 5-chloro-(3,5-dimethylisoxazole-4-yl)-indole, 5-chloro-3-(1-methyl-1H-pyrazol-4-yl)-indole, 5-chloro-3-(1-methyl-1H-pyrazol-5-yl)-indole, 5-chloro-3-(pyrimidin-5-yl)-indole, 5-chloro-3-(3-chloro-4-fluorophenyl)-indole, 5-chloro-3-(thiophen-2-yl)-indole, 5-chloro-3-phenyl-indole, 5-chloro-3-(4-chlorophenyl)-indole, 5-chloro-3-(4-chlorophenyl)-1-methyl-indole, 5-chloro-3-(4-chlorophenyl)-1-isobutyl-indole, 3-(4-chlorophenyl)-5-hydroxy-indole, 3-(4-chlorophenyl)-5-methoxy-indole, 3-(4-chlorophenyl)-5-fluoro-indole, 3-(4-chlorophenyl)-5-(trifluoromethyl)-indole, 3-phenyl-5-(trifluoromethyl)-indole or 3-(3-chloro-4-fluorophenyl)-5-(trifluoromethyl)-indole. In some embodiments, the group comprises(S)—N-(1-(1H-indol-3-yl) butan-2-yl)benzo[d]thiazole-5-carboxamide, (S)—N-(1-(1H-indol-3-yl) butan-2-yl)-5-chloro-1H-indole-2-carboxamide, (R)—N-(1-(1H-indol-3-yl) butan-2-yl)benzo[d]thiazole-5-carboxamide, or (R)—N-(1-(1H-indol-3-yl) butan-2-yl)-5-chloro-1H-indole-2-carboxamide.

The compounds of the presently-disclosed subject matter can be synthesized in view of the information disclosed herein in view of the knowledge of the skilled artisan.

The compounds of the presently-disclosed subject matter can be used for the treatment of cancer, such as brain, colon, prostate, lung, breast, esophageal, pancreatic, skin, Ewing sarcoma, any type of blood cancer etc. MTM derivatives are also neuroprotective and can be used to treat various neuro-diseases, such as Huntington disease, etc.

### Methods of Treatment

In one aspect, the subject technology provides a method for selectively modulating the activity of a target ETS transcription factor in a patient in need thereof, including administering to the patient a therapeutically effective amount of an MTM-OX derivative or a pharmaceutically acceptable salt thereof, as described herein.

In an embodiment relating to this aspect, the subject technology provides a method for selectively modulating the activity of a target ETS transcription factor in a patient with Ewing sarcoma or prostate cancer for example. The method includes administering to the patient a therapeutically effective amount compound as disclosed herein.

In another aspect, the subject technology provides a method of treating a target ETS transcription factor-mediated disease in a patient by administering to the patient a therapeutically effective amount of a compound as disclosed herein, wherein the compound specifically modulates the activity of the ETS transcription factor mediating the disease and wherein the target ETS-mediated disease is Ewing sarcoma, clear-cell sarcoma, myxoid liposarcoma, desmoplastic small round cell tumor, myxoid chondrosarcoma, acute myeloid leukemia, congenital fibrosarcoma, prostate cancer or pancreatic cancer, for example. The following Table lists several ETS transcription factors that may be modulated and associated diseases that may be treated with the subject technology.

ETS Transcription factors and associated diseases.

| Transcription | |
|---|---|
| factor | Disease |
| ETS-1 | Meningioma, invasive carcinoma of the breast, colorectal |
| | carcinoma, pancreatic carcinoma, adenocarcinoma, thyroid |
| | carcinoma, thymoma, angioma |
| ETS-2 | Breast cancer |
| ERG | TMPRSS2: ERG fusion in prostate cancer |
| | EWS-ERG fusion in Ewing Sarcoma |
| | ERG overexpression in AML |
| FLI1 | EWS-FLI1 fusion in Ewing Sarcoma |
| PEA3 | Invasive breast carcinoma |
| ER81 | EWS-ER81 fusion in Ewing sarcoma, prostate carcinoma, |
| | breast carcinoma |
| ELF-1 | Prostate, ovarian and breast cancers, leukemia, |
| | and lymphoma. |
| TEL/ETV6 | TEL fusion protein partners (PDGFbetaR, TRKc, ABL, |
| | and JAK2) in leukemia and fibrosarcoma |
| PU.1/SPI1 | Promyelocytic leukemia, acute myelocytic leukemia |
| Myc | Burkitt lymphoma, B-cell lymphoma, multiple myeloma, |
| | medulloblastoma, neuroblastoma, colorectal, ovarian, and |
| | intestinal cancer |

In general, the compound as disclosed herein can be used for the treatment of a target ETS transcription factor-mediated disease including Ewing sarcoma, clear-cell sarcoma, myxoid liposarcoma, desmoplastic small round cell tumor, myxoid chondrosarcoma, acute myeloid leukemia, congenital fibrosarcoma, prostate cancer, pancreatic cancer, acute myeloid leukemia (AML), acute lymphoblatic leukemia (ALL), Alzheimer's disease (AD), or Down syndrome (DS) or other hyperproliferative disease in which an aberrant activity of a target ETS transcription factor is implicated. A “hyperproliferative disease” includes diseases and conditions that are associated with any sort of abnormal cell growth or abnormal growth regulation, specifically a cancer.

Some of the compounds disclosed herein are more specific than MTM for complexing with a target EST transcription factor and, therefore, inhibiting its activity. The specific or selective the compounds of the subject technology are useful for treating diseases that are mediated by, for example, FLI1 or ERG, such as Ewing sarcoma, clear-cell sarcoma, myxoid liposarcoma, desmoplastic small round cell tumor, myxoid chondrosarcoma, acute myeloid leukemia, congenital fibrosarcoma, prostate cancer, pancreatic cancer, acute myeloid leukemia (AML), acute lymphoblatic leukemia (ALL), Alzheimer's disease (AD), or Down syndrome (DS) or other hyperproliferative disease in which an aberrant activity of a target ETS transcription factor is implicated.

Other hyperproliferative diseases which may be benefited by the methods and compounds of the subject technology include, though it is not limited to, neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; rhabdoid tumors; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In another aspect of the present disclosure, an effective amount of the compounds as disclosed herein or a pharmaceutically acceptable salt thereof is administered to a patient in need of cancer treatment or a neuro-disease, such as Huntington's disease. The compounds or pharmaceutically acceptable salts thereof of the present disclosure can be administered to a patient, e.g., a human patient, in need of such treatment by any route. The compounds or pharmaceutically acceptable salts thereof of the present disclosure can be administered alone or with a pharmaceutically acceptable carrier or excipient.

### Dosage Form and Formulation

A compound as described herein can be administered to a patient in any possible dosage form including, but not limited to ingestible tablets, buccal tablets, troches, capsules, elixirs, suspensions, syrups, wafers, infusion, aqueous liquid and the like. Solutions of the compounds can be prepared in water and mixed with suitable excipients. Under ordinary conditions of storage and use, these preparations can contain a preservative to prevent the growth of microorganisms or retain stabilization of the compound. The pharmaceutical forms suitable for injection include sterile aqueous solutions or dispersions and sterile powders for the extemporaneous preparation of sterile injectable solutions or dispersions. The form should be sterile and should be fluid to the extent it makes injection possible.

A composition containing a compound as disclosed herein can be prepared by known methods, such that an effective quantity of the therapeutic agent is delivered to a subject. Suitable vehicles for such a composition are described, for example, in Remington's Pharmaceutical Sciences (2003) and in the Handbook of Pharmaceutical Additives (compiled by Michael and Irene Ash, Gower Publishing Limited, Aldershot, England (1995)).

In some embodiments, the composition of this disclosure enables sustained, continuous delivery of a compound as disclosed herein to tissues adjacent to or distant from an administration site. The biologically-active agent is capable of providing a local or systemic biological, physiological or therapeutic effect. For example, the compound may act to kill cancer cells or cancer stem cells or to control or suppress tumor growth or metastasis, among other functions.

In some embodiments, the formulations of the present disclosure are administered in an amount effective to provide the desired level of biological, physiological, pharmacological and/or therapeutic effect such as inhibition of a target ETS transcription factor.

The actual dosage amount of a composition of the present disclosure administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical formulations include, for example, at least about 0.1% of an active compound, such as a compound as disclosed herein or pharmaceutically acceptable salt thereof. In other embodiments, the active compound may comprise between about 1% to about 75% of the weight of the unit dosage, or between about 5% to about 50% by weight of the unit dosage, for example, and any specific percentage in between these ranges. In other non-limiting examples, a dose may also comprise from about 0.01 microgram/kg/body weight, about 50 microgram/kg/body weight, about 100 microgram/kg/body weight, about 500 microgram/kg/body weight, about 1 milligram/kg/body weight, about 5 milligram/kg/body weight, about 10 milligram/kg/body weight, about 30 milligram/kg/body weight, about 40 milligram/kg/body weight, about 50 milligram/kg/body weight, about 100 milligram/kg/body weight, or more per administration, and any range or specific amount derivable therein. In non-limiting examples of a derivable range from the numbers listed herein, a range of about 5 microgram/kg/body weight to about 5 milligram/kg/body weight, about 50 microgram/kg/body weight to about 50 milligram/kg/body weight, etc., can be administered.

For a safe and effective dosage, the formulations can be administered at a compound dose of about 0.01 to about 500 mg/m² (body surface)/day, about 0.01 to about 300 mg/m²/day, 0.01 to about 200 mg/m²/day, about 1 to about 200 mg/m²/day about 10 to about 100 mg/m²/day, about 25 to about 100 mg/m²/day or any range derivable therein to a subject such as a human. In certain aspects, the composition may be administered at a dose of about 0.01 to about 200 mg/kg body weight, about 0.01 to about 100 mg/kg body weight, 1 to about 50 mg/kg body weight, about 1 to about 20 mg/kg body weight, about 3 to about 10 mg/kg body weight, about 3 to about 6 mg/kg body weight or any range derivable therein to a subject such as a human. In some embodiments, a formulation of the subject technology may be administered in a dose of 1, 2, 3, 4, 5, 6, 7, 8, 9, 10, 15, 20, 25, 30, 40, 50, 60, 70, 80, 90, 100 mg or more per day. Each liquid dose may be in a volume of 1, 10, 50, 100, 200, 500, 1000 or more μl or ml.

In some embodiments, the pharmaceutical formulation of the subject technology includes an MTM-OX derivative compound as disclosed herein in an amount effective to result in a serum concentration of the compound in the mammal in a range of from 1 nM to 1 mM, particularly 1 nM to 2 μM.

Serum and systemic circulation concentrations of the compound effective to result in the treatment of a target ETS transcription factor-mediated disease may vary depending on a number of factors. Influential variables can include, for example, pKa, solubility or molecular weight of the compound. These properties of a particular MTM-OX derivative may affect how a patient metabolizes the compound, how much of the compound enters and remains in the systemic circulation of the patient, and how effectively the compound treats, prevents or causes regression of the disease, e.g., Ewing sarcoma, tumor or cancer.

Precise amounts of the therapeutic composition also depend on the judgment of the practitioner and are peculiar to each individual. Factors affecting the dose include the physical and clinical state of the patient, the route of administration, the intended goal of treatment (e.g. alleviation of symptoms versus cure) and the potency, stability and toxicity of the particular therapeutic substance.

### Route of Administration

In accordance with the methods of the disclosure, the described composition or formulation of the subject technology may be administered to a subject in a variety of forms depending on the selected route of administration, as will be understood by those skilled in the art. It may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, intratumoral, transepithelial, nasal, intrapulmonary, intrathecal, rectal and topical modes of administration. Parenteral administration may be by continuous infusion over a selected period of time.

### Combination Therapies

In certain embodiments, the compounds, compositions or formulations of the subject technology are administered with a second or additional active agent(s) such as with one or more different MTM-OX derivatives or another anticancer agent. Such therapy can be applied in the treatment of any disease for which treatment with an MTM-OX derivative is contemplated. For example, the disease may be a hyperproliferative disease, such as Ewing sarcoma or prostate cancer.

In certain embodiments, the additional active agent may be a chemotherapeutic agent or a radiation therapy. Examples of chemotherapeutic agents include, but are not limited to, cetuximab (erbitux), herceptin (trastuzumab), fludarabine, cyclophosphamide, rituximab, imatinib, Dasatinib (BMS0354825), cisplatin (CDDP), carboplatin, procarbazine, mechlorethamine, camptothecin, ifosfamide, melphalan, chlorambucil, busulfan, nitrosurea, dactinomycin, daunorubicin, doxorubicin, bleomycin, plicomycin, mitomycin, etoposide (VP 16), tamoxifen, raloxifene, estrogen receptor binding agents, taxol, gemcitabien, navelbine, farnesyl-protein tansferase inhibitors, transplatinum, 5-fluorouracil, vincristin, vinblastin, methotrexate, an analogue or derivative thereof. In certain embodiments, the active or anticancer agent(s) that may be used in combination with an MTM-OX derivative may be fludarabine, cyclophosphamide, rituximab, imatinib or Dasatinib. In a certain aspect, the cancer may be resistant to a particular chemotherapeutic agent, such as fludarabine, cyclophosphamide, rituximab, imatinib or Dasatinib.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

A reference to an element in the singular is not intended to mean “one and only one” unless specifically stated, but rather “one or more.”

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, methylpropynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls. In other embodiments, “alkyl” refers to C₁₋₃ linear hydrocarbon chains, including for example, methyl, ethyl, or propyl groups.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, aryl, substituted aryl, alkoxyl, hydroxyl, aryloxyl, alkoxyl, alkylthio, arylthio, aralkyloxyl, aralkylthio, carboxyl, alkoxycarbonyl, oxo, cycloalkyl, nitro, amino, alkylamino, dialkylamino, sulfate, mercapto, and trimethylsilyl. Thus, as used herein, the term “substituted alkyl” includes alkyl groups, as defined herein, in which one or more atoms or functional groups of the alkyl group are replaced with another atom or functional group.

Further, as used herein, the terms alkyl and/or “substituted alkyl” include an “allyl” or an “allylic group.” The terms “allylic group” or “allyl” refer to the group —CH₂HC═CH₂ and derivatives thereof formed by substitution. Thus, the terms alkyl and/or substituted alkyl include allyl groups, such as but not limited to, allyl, methylallyl, di-methylallyl, and the like. The term “allylic position” or “allylic site” refers to the saturated carbon atom of an allylic group. Thus, a group, such as a hydroxyl group or other substituent group, attached at an allylic site can be referred to as “allylic.”

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine, benzophenone, furan, thiophene, pyrrole, pyran, pyridine, imidazole, benzimidazole, benzothiazole, isothiazole, isoxazole, pyrazole, pyrazine, triazine, pyrimidine, quinoline, isoquinoline, indole, carbazole, and the like. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, CF₃, and —NR′R′, wherein R′ and R′ can each be independently hydrogen, alkyl, substituted alkyl, aryl, substituted aryl, and aralkyl. Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

“Alkene” or “Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

“Alkyne” refers to a functional group consisting of two carbon atoms bonded by a triple bond (—C≡C—). An alkyne compound would have the formula R—C≡C—R, where R is a hydrogen atom or an alkyl group. A terminal alkyne compound would have the formula R—C≡C—H, where R is an alkyl group. A cyclic alkyne compound would have the alkyne functionality in a cyclic structure. Examples include, but are not limited to ethyne or acetylene and propyne.

As used herein, the term “acyl” refers to an organic acid group wherein the —OH of the carboxyl group has been replaced with another substituent (i.e., as represented by RCO—, wherein R is an alkyl or an aryl group as defined herein). As such, the term “acyl” specifically includes arylacyl groups, such as an acetylfuran and a phenacyl group. Specific examples of acyl groups include acetyl and benzoyl. The acyl group can be optionally substituted (a “substituted acyl”) with one or more acyl group substituents, which can be the same or different, wherein “acyl group substituent” includes aryl, amino acid, and amino acid dipeptide. In some embodiments, the term “acyl” refers to an organic acid group wherein the OH of the carboxyl group has been replaced with another substituent (i.e., as represented by RCO—, wherein R is an alkyl or an aryl group as defined herein). As such, the term “acyl” specifically includes acetyl group.

As used herein, the term “amino acid” refers to a functional group or component thereof that is derived from an amino acid molecule, such as, for example, tryptophan, phenylalanine, alanine, or tyrosine. The term “amino acid dipeptide” refers to a functional group or component thereof that is derived from a peptide including two amino acid molecules, such as, for example, phenylalanine-tryptophan or tryptophan-tryptophan. An amino acid group or amino acid dipeptide group can be optionally substituted. In some embodiments, an amino acid group or amino acid dipeptide group can be derived from an amino acid molecule, such as, for example, tryptamine (Tra), methyl tryptophan, or tryptophan methyl ester derived from tryptophan,

As used herein, a “target ETS transcription factor” refers to a transcription factor, which comprises a DNA-binding domain (DBD) having an amino acid sequence that is at least 85%, 90%, 95%, 98%, or 99% identical to SEQ ID NO: 1 or SEQ ID NO: 2, over the entire length of either of these sequences.

As used herein the term “modulator,” “modulating,” or “modulate” in connection with the target ETS transcription factor of the subject technology refers to any agent that has a functional effect on the transcription factor, including positively or negatively affecting its binding to a DNA substrate, positively or negatively affecting the formation and/or stability of a complex formed between the transcription factor and its oligonucleotide substrate, positively or negatively affecting its function in causing the transcription of its oligonucleotide substrate.

As used herein, the term “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “variant” in relation to the amino acid sequence of the ETS transcription factors refers to a naturally occurring allelic variant of the ETS transcription factors such as those shown in SEQ ID NO: 3 and SEQ ID NO: 4, which includes any substitution of, variation of, modification of, replacement of, deletion of or addition of one (or more) amino acids provided the resultant ETS transcription factor has a transcription factor activity and has a DNA binding domain that is at least 85%, 90%, 95%, 98%, or 99% identical to SEQ ID NO: 1 or SEQ ID NO: 2, over the entire length of either of these sequences. For example, a variant of ETS transcription factor may have at least 50%, or at least 60%, or at least 70% sequence identity with the ETS transcription factors such as those shown in SEQ ID NO: 3 and SEQ ID NO: 4, over the entire length of the sequence, provided that the variant has a transcription factor activity and has a DNA binding domain that is at least 85%, at least 90%, at least 95% or at least 98% identical to SEQ ID NO: 1 or SEQ ID NO: 2, over the entire length of either of these sequences.

The terms “percentage of sequence identity” or “percentage homology” and any equivalent terms are used interchangeably herein to refer to comparisons among polynucleotides and polypeptides, and are determined by comparing two optimally aligned sequences over a comparison window, wherein the portion of the oligonucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Identity is evaluated using any of the variety of sequence comparison algorithms and programs known in the art. Such algorithms and programs include, but are by no means limited to, TBLASTN, BLASTP, FASTA, TFASTA, CLUSTALW, FASTDB [Pearson and Lipman, (1988), Proc. Natl. Acad. Sci. USA 85 (8): 2444-2448; Altschul et al., (1990), J. Mol. Biol. 215 (3): 403-410; Thompson et al. (1994), Nucleic Acids. Res. 22 (2): 4673-4680; Higgins et al., (1996), Meth. Enzymol. 266:383-402; Altschul et al., (1993), Nature Genetics 3:266-272; Brutlag et al. (1990) Comp. App. Biosci. 6:237-24], the disclosures of which are incorporated by reference in their entireties. In an embodiment, protein and nucleic acid sequence identities are evaluated using the Basic Local Alignment Search Tool (“BLAST”) which is well known in the art [e.g., Karlin and Altschul, (1990), Proc. Natl. Acad. Sci. USA 87:2267-2268; Altschul et al., (1997), Nuc. Acids Res. 25:3389-3402] the disclosures of which are incorporated by reference in their entireties.

As used herein, an “oligonucleotide substrate” in reference to a substrate of a target ETS transcription factor refers to an oligonucleotide which comprises a target ETS transcription factor binding site. An oligonucleotide substrate can be single-stranded, double-stranded, or a hairpin. Preferably, an oligonucleotide substrate is double stranded. An oligonucleotide substrate can be DNA, RNA or a chimeric (comprising both deoxy and ribose nucleotides) or comprise one or more oligonucleotide modifications described herein.

As used herein, the term “transcription factor binding site” refers to a nucleic acid sequence that is recognized and bound by a transcription factor and mediates the transactivation of a reporter gene in response to that binding. Without limitations, a transcription binding site can be from any of various species including human, mouse, rat, guinea pig and the like. In some embodiments, the transcription factor binding site is a target ETS binding site such as a FLI1 binding site or an ERG binding site.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9): 1726-1732).

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

Where reference is made to a URL or other such identifier or address, it understood that such identifiers can change and particular information on the internet can come and go, but equivalent information can be found by searching the internet. Reference thereto evidences the availability and public dissemination of such information.

The presently-disclosed subject matter is further illustrated by the following specific but non-limiting examples. The following examples may include compilations of data that are representative of data gathered at various times during the course of development and experimentation related to the present invention.

### EXAMPLES

### Example 1—Activity

Exemplary compounds of the presently-disclosed subject matter were assessed for activity, including clearance, potency, and selectivity. Results from studies are compiled in Tables 2-4.

Desirable clearance was identified at lower values of mL hr⁻¹kg⁻¹ (e.g., about ≤10 mL hr⁻¹ kg⁻¹), desirable potency was identified at lower values of IC₅₀ (e.g., about ≤10 nM in TC32 cells), and desired selectivity was identified at higher ratios of IC₅₀ in PC3/IC₅₀ in TC32 (e.g. about >10).

TABLE 2

| | | CL | IC₅₀ | | Selectivity |
|---|---|---|---|---|---|
| | log P | (mL hr⁻¹kg⁻¹) | TC32 (nM) | PC2 (nM) | Ratio |
| YL-B152 | 2.19 | 44 | 8.34 | 17.6 | 2.1 |
| YL-B158 | 1.28 | NT | 35.6 | 300 | 8.4 |
| YL-B160 | 1.30 | NT | 86.1 | 1130 | 13.1 |
| YL-B162 | −0.56 | NT | 2840 | 3000 | 1.1 |
| YL-B166 | 1.56 | NT | 2.33 | 32.3 | 13.9 |
| YL-B167 | 2.83 | NT | 15.1 | 48.4 | 3.2 |
| YL-B168 | 1.62 | 43 | 2.15 | 19.5 | 9.1 |
| YL-B169 | 2.37 | 28 | 1.16 | 20.6 | 17.8 |
| YL-B170 | 1.02 | NT | 70.7 | 365 | 5.2 |
| YL-B171 | 2.93 | NT | 4.62 | 10.7 | 2.3 |
| YL-B175 | 2.54 | NT | 1.2 | 1.5 | 1.2 |
| YL-B176 | 3.49 | 21 | 13.9 | 178 | 12.8 |
| YL-B179 | −0.02 | NT | 679 | 3000 | 4.4 |
| YL-C09 | 1.9 | NT | 1.96 | 5.65 | 2.9 |
| YL-C20 | 2.10 | NT | 69.8 | 1020 | 14.6 |
| YL-C39 | 2.56 | NT | 864 | 3000 | 3.5 |
| YL-C51 | 4.19 | 15 | 456 | 2790 | 6.1 |
| YL-C52 | 1.87 | NT | 6.41 | 8.07 | 1.3 |
| YL-C53 | 3.26 | NT | 23.1 | 59.8 | 2.6 |
| YL-C54 | 4.34 | NT | 40.2 | 130 | 3.2 |
| YL-C55 | 3.26 | NT | 21.4 | 64.4 | 3.0 |
| YL-C56 | 4.34 | NT | 26.2 | 82.8 | 3.2 |
| YL-C61 | 1.12 | NT | 64.3 | 214 | 3.3 |
| YL-C67 | 4.56 | 10 | 116 | 1230 | 10.6 |
| YL-C82 | 4.21 | 23 | 20.2 | 515 | 25.5 |
| YL-C95 | 4.69 | 26 | 5.31 | 266 | 50 |
| YL-C98 | 4.6 | NT | 24.2 | 132 | 5.5 |
| YL-100 | 6.21 | NT | 97 | 1500 | 15.5 |
| YL-C102 | 2.26 | NT | 7.4 | 43 | 5.8 |
| YL-C104 | 1.77 | 31 | 5.63 | 23 | 4.1 |
| YL-C106 | 3.3 | 19 | 17.2 | 202 | 11.7 |
| YL-C108 | 1.44 | 24 | 6.12 | 40.3 | 6.6 |
| YL-C110 | 2.56 | NT | 2.18 | 7.1 | 3.3 |
| YL-C114 | 3.58 | 28 | 3.04 | 15.9 | 5.2 |
| YL-C116 | 4.19 | NT | 193 | 1560 | 8.1 |
| YL-C118 | 3.64 | 28 | 36.6 | 582 | 15.9 |
| YL-C120 | 3.04 | 25 | 7.6 | 31.4 | 4.1 |
| YL-C122 | 1.73 | 41 | 18.5 | 124 | 6.7 |
| YL-C124 | 3.06 | NT | 22.4 | 169 | 7.5 |

TABLE 3

| | | CL | IC₅₀ | | Selectivity |
|---|---|---|---|---|---|
| | log P | (mL hr⁻¹kg⁻¹) | TC32 (nM) | PC2 (nM) | Ratio |
| YL-B176 | 3.49 | 21 | 13.9 | 178 | 13 |
| YL-C66 | 3.84 | 12 | 22 | 283 | 13 |
| YL-C67 | 4.56 | 10 | 116 | 1230 | 11 |
| YL-C68 | 4.54 | | 52.8 | 430 | 8 |
| YL-C82 | 4.21 | 23 | 12 | 218 | 18 |
| YL-C106 | 3.3 | 19 | 17 | 202 | 12 |
| UL-C116 | 4.19 | ND | 192.2 | 1465 | 7.6 |

TABLE 4

| | | | | | CL | |
|---|---|---|---|---|---|---|
| | | IC₅₀ | | In vitro | (mL hr⁻¹ | PK |
| | log P | TC32 (nM) | PC2 (nM) | Selectivity | kg⁻¹) | Improvement |
| YL-B152 | 2.2 | 8.3 | 17.6 | 2.1 | 44 | 35.9 |
| YL-B176 | 3.5 | 13.9 | 178 | 12.8 | 21 | 75.1 |
| YL-C66 | 3.8 | 22.0 | 283 | 12.9 | 12 | 131.5 |
| YL-C67 | 4.6 | 116 | 1230 | 10.6 | 10 | 157.8 |
| YL-C82 | 4.2 | 20.2 | 515 | 25.5 | 23 | 68.6 |
| YL-C106 | 3.3 | 17.2 | 202 | 11.7 | 19 | 83.1 |

### Example 2—Ewing Sarcoma In Vitro Cytotoxicity

Exemplary compounds of the presently-disclosed subject matter were assessed for cytotoxicity (Table 5) in Ewing sarcoma (TC32) and non-Ewing (PC3 prostate cancer). Select compounds with GI₅₀ in TC32 of 30 or less were evaluated further in an expanded panel of Ewing and non-Ewing cell lines (Table 6) to evaluate the relative cytotoxicity in cells expressing EWS-FLI1. Cells (1000-5000/well) were seeded in 96-well plates and allowed to attach and grow for 24 hr prior to adding compounds in half-log increments from 0.3 nM-10 μM. Cell viability was assessed after 72 hr continuous incubation with compounds using resazurin fluorescence. GI50 values were estimated using GraphPad Prism.

TABLE 5
GI₅₀ values estimated by cytotoxicity assay in vitro (72 hr).

| GI₅₀, nM | TC32 | PC3 |
|---|---|---|
| MTM | 10 | 11 |
| AK-B11 | 495 | 869 |
| AK-B16 | 447 | 679 |
| AK-B6 | 202 | 414 |
| YL-B121 | 449 | 1000< |
| YL-B168 | 30 | 65 |
| YL-B169 | 18 | 34 |
| YL-B171 | 26 | 49 |
| YL-B176 | 117 | 304 |
| YL-B62 | 399 | 1000< |
| YL-C09 | 17 | 19 |
| YL-C102 | 49 | 334 |
| YL-C104 | 34 | 133 |
| YL-C106 | 99 | 236 |
| YL-C108 | 34 | 126 |
| YL-C110 | 14 | 27 |
| YL-C114 | 43 | 152 |
| YL-C118 | 232 | 417 |
| YL-C120 | 36 | 66 |
| YL-C122 | 162 | 477 |
| YL-C36 | 1000< | 1000< |
| YL-C51 | 1000< | 1000< |
| YL-C66 | 191 | 630 |
| YL-C67 | 902 | 1000< |
| YL-C68 | 777 | 1000< |
| YL-C76 | 702 | 1000< |
| YL-C81 | 1000< | 71 |
| YL-C82 | 356 | 805 |
| YL-C95 | 608 | 1000< |

TABLE 6
GI₅₀ values in expanded panels of Ewing (EWS) and non-Ewing cell lines.
Among Ewing cells, 5838 cells express EWS-ERG. BL1395 cells
are of lymphocyte origin and HepG2 of hepatic origin.

| EWS cells | TC32 | TC71 | 5838 | A673 | RDES | SK.ES1 | ES3 | ES4 | ES7 | ES8 | Average |
|---|---|---|---|---|---|---|---|---|---|---|---|
| MTM | 10.4 | 10.3 | 11.2 | 9.1 | 10.6 | 10.2 | 12.8 | 7.3 | 10.0 | 17.4 | 10.9 |
| YL-B168 | 30.3 | 258.3 | 40.2 | 34.5 | 32.9 | 39.2 | 56.7 | 30.9 | 76.9 | 68.8 | 66.9 |
| YL-B169 | 18.4 | 152.5 | 30.2 | 26.0 | 29.1 | 24.4 | 38.4 | 25.1 | 25.6 | 68.7 | 43.8 |
| YL-B171 | 25.6 | 19.5 | 37.1 | 51.0 | 32.7 | 35.4 | 31.5 | 20.4 | 26.7 | 36.7 | 31.7 |
| YL-C09 | 17.0 | 11.5 | 27.5 | 38.1 | 16.8 | 18.0 | 19.2 | 16.6 | 14.5 | 31.5 | 21.1 |
| YL-C110 | 14.3 | 10.7 | 26.6 | 33.5 | 13.3 | 18.8 | 18.8 | 10.1 | 13.6 | 16.6 | 17.6 |

| Non EWS | | | | | | | | | | |
|---|---|---|---|---|---|---|---|---|---|---|
| cells | PC3 | BL1395 | A549 | DU145 | H460 | Hela | HepG2 | LNCap | PANC1 | Average |
| MTM | 11.3 | 9.9 | 54.0 | 16.4 | 6.8 | 28.4 | 28.4 | 82.7 | 22.3 | 28.9 |
| YL-B168 | 65.2 | 49.9 | 357.5 | 90.5 | 115.9 | 470.0 | 1000< | 275.1 | 105.0 | 191.1 |
| YL-B169 | 34.0 | 31.3 | 557.6 | 49.9 | 78.7 | 175.9 | 306.3 | 239.2 | 62.1 | 170.6 |
| YL-B171 | 49.3 | 51.5 | 113.8 | 32.5 | 69.5 | 104.6 | 202.0 | 116.8 | 138.4 | 97.6 |
| YL-C09 | 19.3 | 24.1 | 22.6 | 19.0 | 21.5 | 27.1 | 80.1 | 63.3 | 33.4 | 34.5 |
| YL-C110 | 26.8 | 49.4 | 46.7 | 16.2 | 33.2 | 102.1 | 466.4 | 44.3 | 87.5 | 96.9 |

### Example 3—Pharmacokinetics

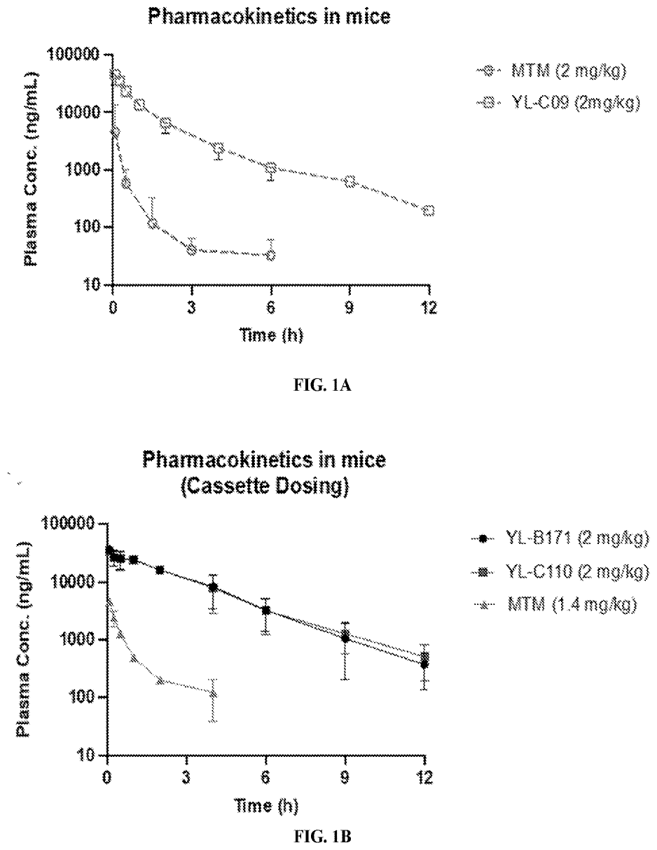

Pharmacokinetics of exemplary compounds of the presently-disclosed subject matter were studied in mice following bolus IV dosing. Groups of three mice were administered the compound(s) and blood samples were collected via saphenous bleed. Plasma was separated by centrifugation and extracted to quantitate analytes by LC/MS/MS. Sample analysis followed upon the system meeting system suitability criteria customary for fit for purpose analytical methods. FIG. 1A includes data comparing YL-C09 to mithramycin (MTM) dosed separately. FIG. 1B includes data for YL-B171, YL-C110 and MTM dosed in cassette format.

### Example 4—Rhabdomyosarcoma In Vitro Cytotoxicity

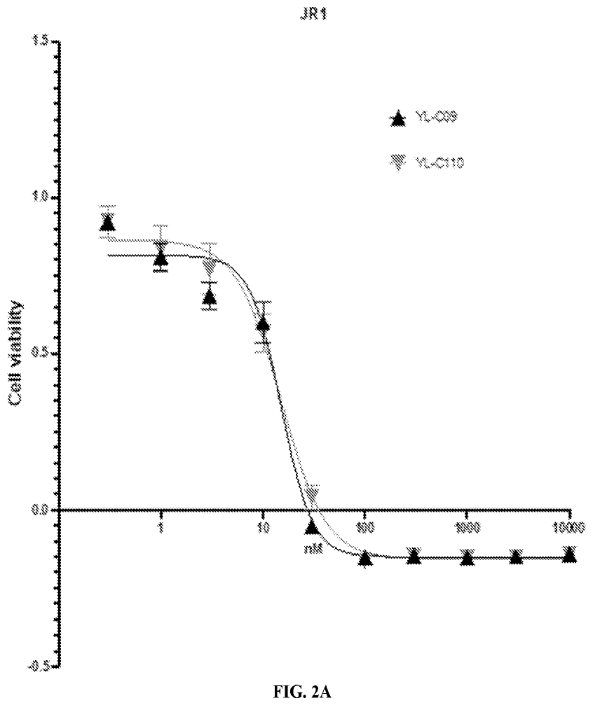

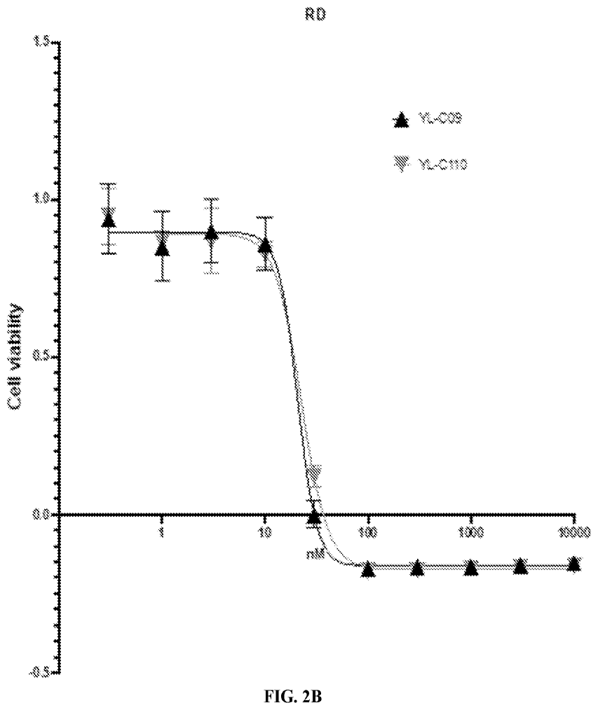

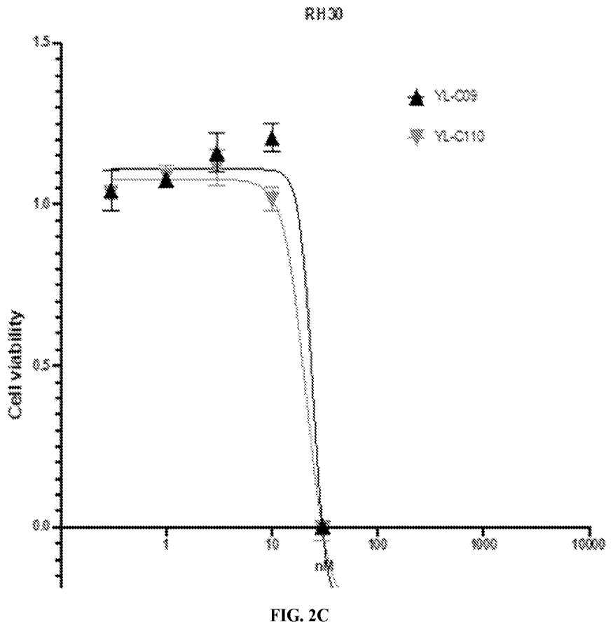

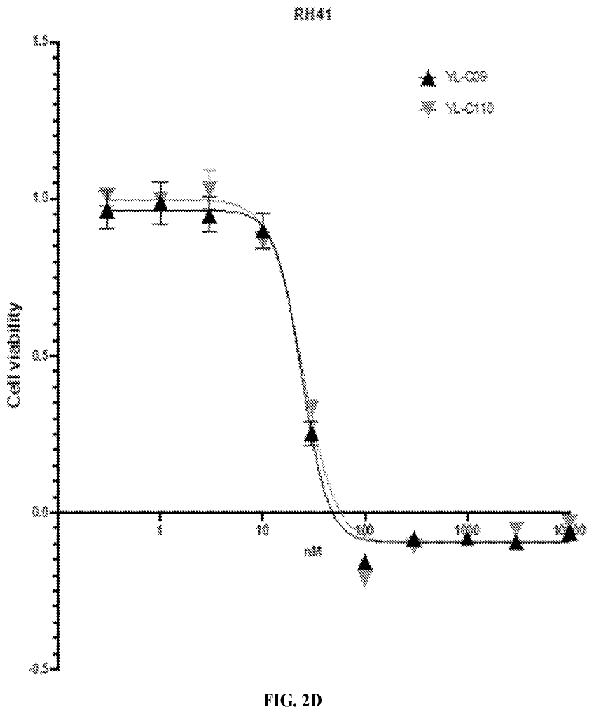

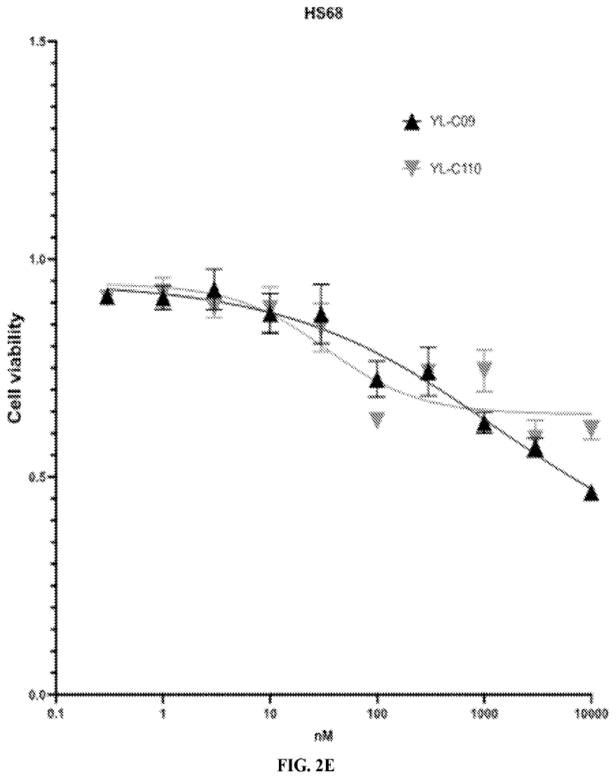

YL-C09 and YL-C110 were tested for cytotoxicity (GI₅₀, 72 hr resazurin assay) in rhabdomyosarcoma cells of embryonal (JR1 (FIG. 2A), RD (FIG. 2B) cell lines) and alveolar (RH30 (FIG. 2C), RH41 (FIG. 2D) cell lines) histologies. A fibroblast cell line (HS68 (FIG. 2E)) was used as a non-tumor control. The area under the concentration-efficacy curve (AUC; possible range of 0-20000) was used to estimate an alternate metric to GI₅₀ for cases of shallow efficacy (e.g., the limited effect in HS68 cells). Figure X2 shows the GI₅₀ of YL-C09 and YL-C110 ranges between 15-25 nM in the four cell lines tested while the effect in HS68 cells is nominal (FIG. 2A-2E, Table 7).

TABLE 7

| Cell Line | Compound | GI₅₀ (nM) | 95% CL | AUC |
|---|---|---|---|---|
| HS68 | YL-C09 | 1241 | >272.9 | 15517 |
| | YL-C110 | 33.56 | 13.16-86.14 | 16245 |
| JR1 | YL-C09 | 15.03 | 11.79-17.49 | 8554 |
| | YL-C110 | 14.95 | 10.84-16.56 | 8535 |
| RD | YL-C09 | 20.69 | >16.25 | 8443 |
| | YL-C110 | 22.45 | 18.76-24.61 | 8367 |
| RH30 | YL-C09 | 24.26 | >22.15 | 7400 |
| | YL-C110 | 21.03 | >20.03 | 7413 |
| RH41 | YL-C09 | 24.13 | 21.39-27.76 | 9239 |
| | YL-C110 | 24.74 | 21-71-27.82 | 9477 |

### Example 5—Molecular Effects of YL-C09 and YL-C110

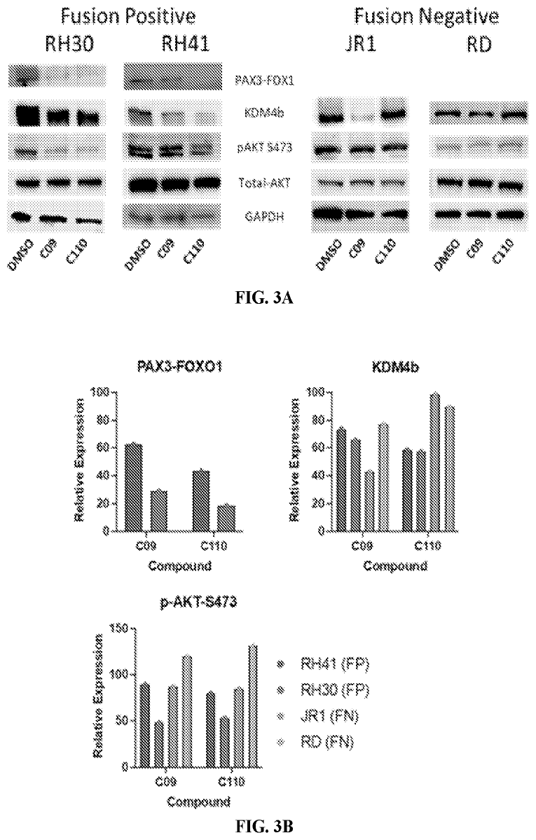

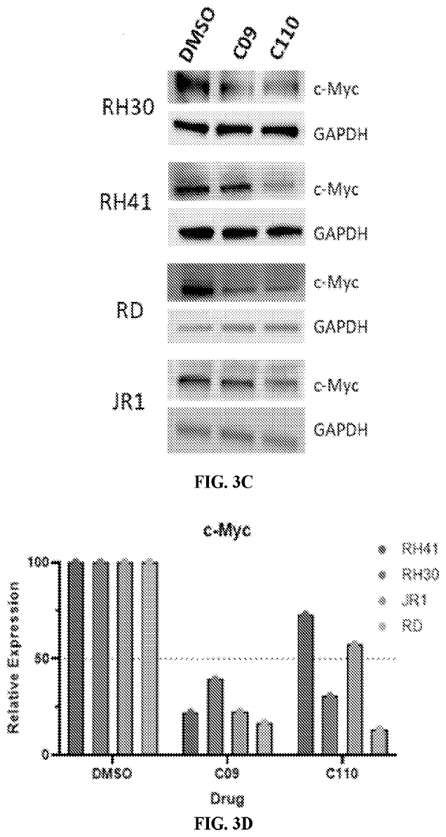

To assess the effect of compounds on known molecular effectors of rhabdomyosarcoma we treated 0.5×10⁶ cells with 10 nM of each compound for 24 hr and subjected cell lysates to western blotting as indicated in FIG. 3A and FIG. 3B (semiquantitative estimation of compounds' effect based on band intensities). All values were normalized to GAPDH and plotted relative to DMSO (vehicle) treated cells which was set to 100 percent. Alveolar histology cell lines (RH30 and RH41; PAX3-FOXO1, fusion-positive) treated with either compound had decreased expression of PAX3-FOXO1 and downstream targets (ALK, FGFR4, and MET; data not shown). Additional effects may be mediated through interaction with the histone demethylase KDM4B, known to play an important role in the epigenetic programming of rhabdomyosarcoma. Given the potent activity of both analogues in alveolar and embryonal cell lines (JR1, RD; fusion-negative), we investigated whether the oncogene c-Myc was affected with treatment (FIG. 3C and FIG. 3D). Upon treatment with compounds at 10 nM for 24 hr, c-Myc expression was decreased suggesting that this effect may contribute to the observed cytotoxicity.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Wohlert, S.; Künzel, E.; Machinek, R.; Mendez, C.; Salas, J.; Rohr, J. The structure of mithramycin reinvestigated. J. Nat. Prod. 1999, 62, 119-121.
2. Rohr, J.; Méndez, C.; Salas, J. A. The biosynthesis of aureolic acid group antibiotics. Bioorg. Chem. 1999, 27, 41-54.
3. Kofman, S., Perlia, C. P, Economou, S. G. Mithramycin in the treatment of metastatic ewing's sarcoma. Cancer 1973, 31, 889-893.; b) Balamuth, N., Womer, R. B.: Ewing's sarcoma, Lancet Oncol. 2010, 11, 184-192.
4. (a) Kofman, S.; Eisenstein, R. Mithramycin in the treatment of disseminated cancer. Cancer Chemother. Rep. 1963, 32, 77-96.; (b) Kofman, S.; Medrek, T. J.; Alexander, R. W. Mithramycin in the treatment of embryonal cancer. Cancer 1964, 17, 938-948.
5. Delattre, O.; Zucman, J.; Plougastel, B.; Desmaze, C.; Melot, T.; Peter, M.; Kovar, H.; Joubert, I.; de Jong, P.; Rouleau, G. Gene fusion with an ETS DNA-binding domain caused by chromosome translocation in human tumours. Nature 1992, 359, 162.
6. May, W. A.; Arvand, A.; Thompson, A. D.; Braun, B. S.; Wright, M.; Denny, C. T. EWS/FLI1-induced manic fringe renders NIH 3T3 cells tumorigenic. Nat. Genet. 1997, 17, 495-497.
7. Tomlins, S. A., Rhodes, D. R., Perner, S., Dhanasekaran, S. M., Mehra, R., Sun, X. W., Varambally, S., Cao, X., Tchinda, J., Kuefer, R. and Lee, C. Recurrent fusion of TMPRSS2 and ETS transcription factor genes in prostate cancer. Science, 2005, 310,644-648.
8. Sastry, M.; Patel, D. J. Solution structure of the mithramycin dimer-DNA complex. Biochemistry 1993, 32, 6588-6604.
9. Remsing, L. L.; González, A. M.; Nur-e-Alam, M.; Fernández-Lozano, M. J.; Braña, A. F.; Rix, U.; Oliveira, M. A.; Méndez, C.; Salas, J. A.; Rohr, J. Mithramycin SK, a novel antitumor drug with improved therapeutic index, mithramycin SA, and demycarosyl-mithramycin SK: three new products generated in the mithramycin producer streptomyces argillaceus through combinatorial biosynthesis. J. Am. Chem. Soc. 2003, 125, 5745-5753.
10. Scott, D.; Chen, J. M.; Bae, Y.; Rohr, J. Semi-synthetic mithramycin SA derivatives with improved anti-cancer activity. Chem. Biol. Drug. Des. 2013, 81, 615-624.
11. Leggas, M.; Eckenrode, J.; Mitra, P.; Jha, J.; Salem, S.; Mandal, A.; Thorson, J.; Rohr, J. [abstract]. In: Proceedings of the AACR-NCI-EORTC international conference: molecular targets and cancer therapeutics; 2017 Oct. 26-30; Philadelphia, PA. Philadelphia (PA): AACR; Mol Cancer Ther. 2018, 17 (1 Suppl): Abstract nr B043.
12. Hou, C.; Weidenbach, S.; Cano, K. E.; Wang, Z.; Mitra, P.; Ivanov, D. N.; Rohr, J.; Tsodikov, O. V. Structures of mithramycin analogues bound to DNA and implications for targeting transcription factor FLI1. Nucleic Acids Res. 2016, 44, 8990-9004.
13. Alqahtani, N.; Porwal, S. K.; James, E. D.; Bis, D. M.; Karty, J. A.; Lane, A. L.; Viswanathan, R. Synergism between genome sequencing, tandem mass spectrometry and bio-inspired synthesis reveals insights into nocardioazine B biogenesis. Org. Biomol. Chem. 2015, 13, 7177-7192.
14. Cardoso, A. S. P.; Marques, M. M. B.; Srinivasan, N.; Prabhakar, S.; Lobo, A. M.; Rzepa, H. S. Studies in sigmatropic rearrangements of N-prenylindole derivatives-a formal enantiomerically pure synthesis of tryprostatin B. Org. Biomol. Chem. 2006, 4, 3966-3972.
15. Loach, R. P.; Fenton, O. S.; Amaike, K.; Siegel, D. S.; Ozkal, E.; Movassaghi, M. Derivatization of C3-alkylindoles including tryptophans and tryptamines. J. Org. Chem. 2014, 79, 11254-11263.
16. Partridge, B. M.; Hartwig, J. F. Sterically controlled iodination of arenes via iridium-catalyzed C—H borylation. Org. Lett. 2012, 15, 140-143.
17. Feng, Y.; Holte, D.; Zoller, J.; Umemiya, S.; Simke, L. R.; Baran, P. S. Total synthesis of erruculogen and fumitremorgin a enabled by ligand-controlled CH borylation. J. Am. Chem. Soc. 2015, 137, 10160-10163.
18. Jia, Y.; Zhu, J. Palladium-catalyzed, modular synthesis of highly functionalized indoles and tryptophans by direct annulation of substituted o-haloanilines and aldehydes. J. Org. Chem. 2006, 71, 7826-7834.
19. Kokotos, G.; Padrón, J. M.; Martín, T.; Gibbons, W. A.; Martín, V. S. A general approach to the asymmetric synthesis of unsaturated lipidic α-amino acids. The first synthesis of α-aminoarachidonic acid. J. Org. Chem. 1998, 63, 3741-3744.
20. Bi, W.; Bi, Y.; Xue, P.; Zhang, Y.; Gao, X.; Wang, Z.; Li, M.; Baudy-Floc′h, M.; Ngerebara, N.; Li, X. Novel β-carboline-tripeptide conjugates attenuate mesenteric ischemia/reperfusion injury in the rat. Eur. J. Med. Chem. 2011, 46, 2441-2452.
21. Coste, A.; Toumi, M.; Wright, K.; Razafimahaléo, V.; Couty, F.; Marrot, J.; Evano, G. Copper-catalyzed cyclization of iodo-tryptophans: A straightforward synthesis of pyrroloindoles. Org. Lett. 2008, 10, 3841-3844.
22. Cozett, R. E.; Venter, G. A.; Gokada, M. R.; Hunter, R. Catalytic enantioselective acyl transfer: the case for 4-PPY with a C-3 carboxamide peptide auxiliary based on synthesis and modelling studies. Org. Biomol. Chem. 2016, 14, 10914-10925.
23. Choi, J. Y.; Calvet, C. M.; Gunatilleke, S. S.; Ruiz, C.; Cameron, M. D.; McKerrow, J. H.; Podust, L. M.; Roush, W. R. Rational development of 4-aminopyridyl-based inhibitors targeting Trypanosoma cruzi CYP51 as anti-chagas agents. J. Med. Chem. 2013, 56, 7651-7668.
24. Osgood, C. L.; Maloney, N.; Kidd, C. G.; Kitchen-Goosen, S.; Segars, L.; Gebregiorgis, M.; Woldemichael, G. M.; He, M.; Sankar, S.; Lessnick, S. L.; Kang, M.; Smith, M.; Turner, L.; Madaj, Z. B.; Winn, M. E.; Núñez, L. E.; González-Sabín, Z.; Helman, L. J.; Morís, F.; Grohar, P. J. Identification of mithramycin analogues with improved targeting of the EWS-FLI1 transcription factor. Clin. Cancer Res. 2016, 22, 4105-4118.
25. Garcia-Aragoncillo, E., J. Carrillo, E. Lalli, N. Agra, G. Gomez-Lopez, A. Pestana, and J. Alonso. “DAX1, a direct target of EWS/FLI1 oncoprotein, is a principal regulator of cell-cycle progression in ewing's tumor cells.” Oncogene 2008, 27, 6034-6043.
26. Grohar, P. J.; Woldemichael, G. M.; Griffin, L. B.; Mendoza, A.; Chen, Q.-R.; Yeung, C.; Currier, D. G.; Davis, S.; Khanna, C.; Khan, J. Identification of an inhibitor of the EWS-FLI1 oncogenic transcription factor by high-throughput screening. J. Natl. Cancer Inst. 2011, 103, 962-978.
27. Mitra, P., Eckenrode, J. M., Mandal, A., Jha, A. K., Salem, S. M., Leggas, M., & Rohr, J. Development of mithramycin analogues with increased selectivity toward ETS transcription factor expressing cancers. Journal of medicinal chemistry, 2018, 61 (17), 8001-8016.
28. Tevyashova, A., Zbarsky, E., Balzarini, J. et al. Modification of the antibiotic olivomycin I at the 2′-keto group of the side chain. Novel derivatives, antitumor and topoisomerase I-poisoning activity. J Antibiot 2009, 62, 37-41.
29. Liu, Y., Eckenrode, J. M., Zhang, Y., Zhang, J., Hayden, R. C., Kyomuhangi, A., . . . & Thorson, J. S. Mithramycin 2′-oximes with improved selectivity, pharmacokinetics, and Ewing sarcoma antitumor efficacy. Journal of medicinal chemistry, 2020, 63 (22), 14067-14086.
30. U.S. Pat. No. 9,447,135 to Rohr, J. T, et al., “Semi-Synthetic Mithramycin Derivatives with Anti-Cancer Activity.”
31. International Patent Application Publication No. WO 2010126626 to deLong, et al., “Preparation of isoquinolinylamide derivatives for use as dual-action monoamine transport and kinase inhibitors.”
32. U.S. patent application Ser. No. 16/670,707, issued as U.S. Pat. No. 11,466,045 to Thorson, et al. for “Mithramycin Oxime Derivatives Having Increased Selectivity and Anti-Cancer Activity.”
33. U.S. patent application Ser. No. 17/963,537 to Thorson, et al., for “Mithramycin Oxime Derivatives Having Increased Selectivity and Anti-Cancer Activity.”

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A compound having the following formula: 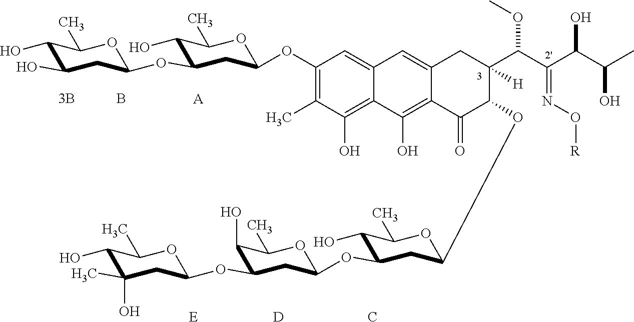
or a pharmaceutically acceptable salt thereof,
wherein R is

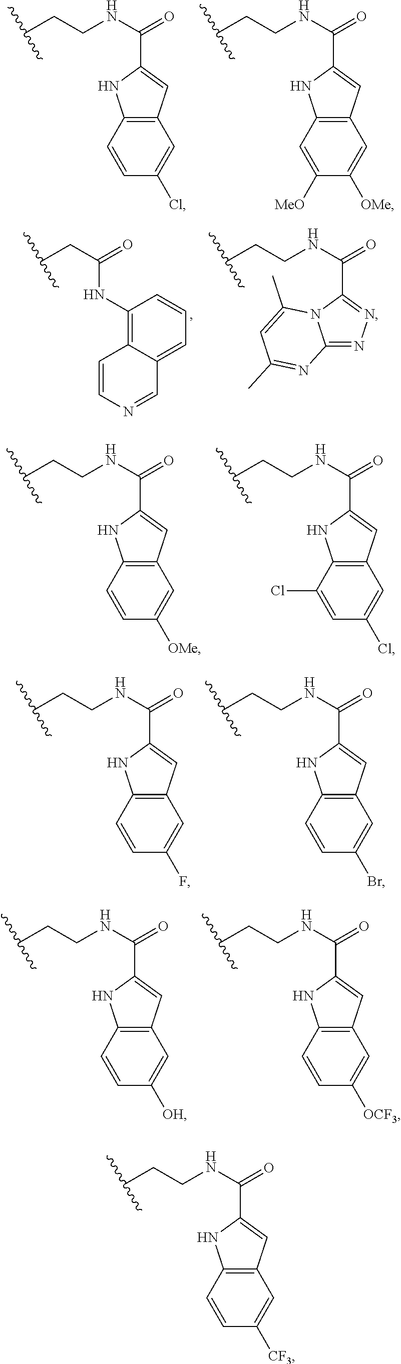

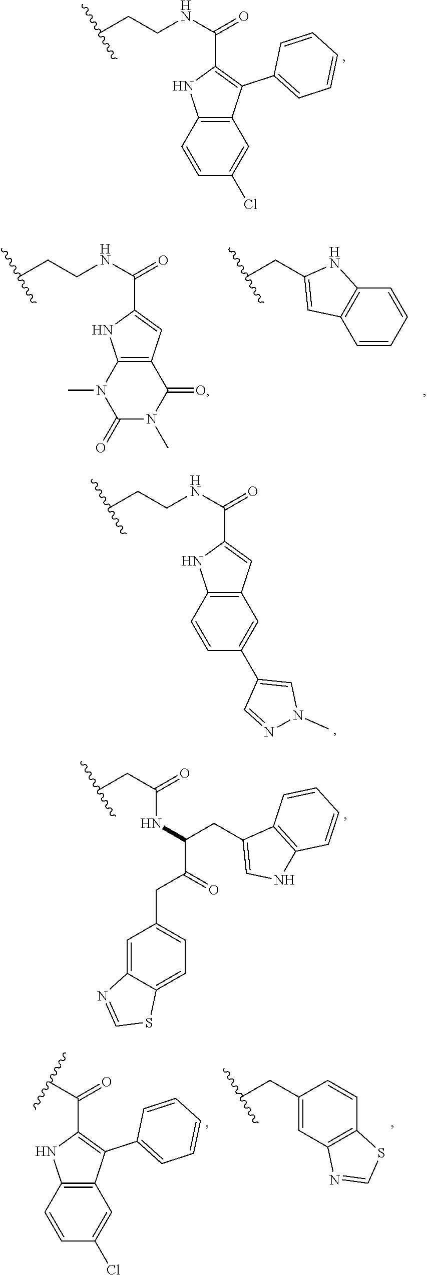

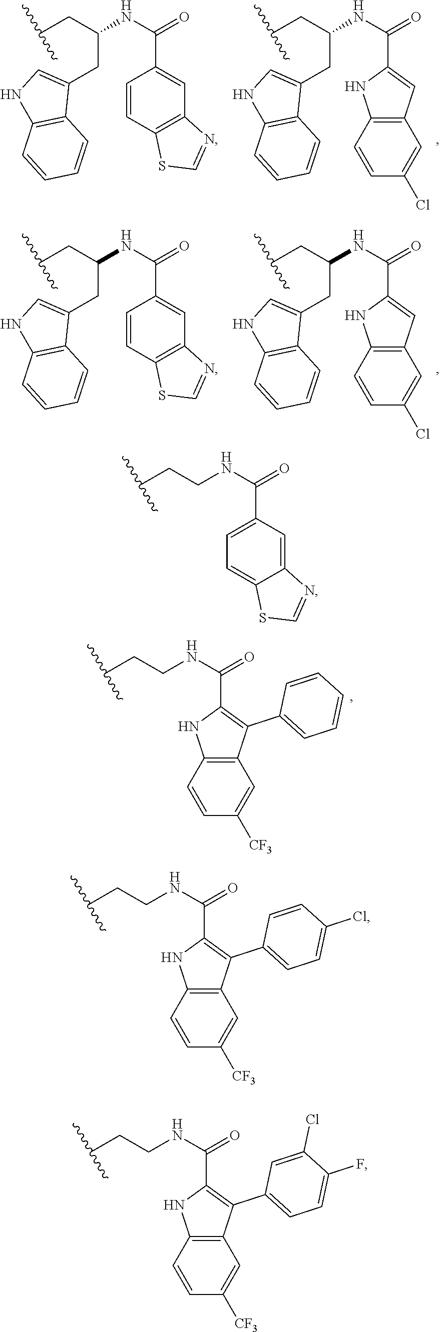

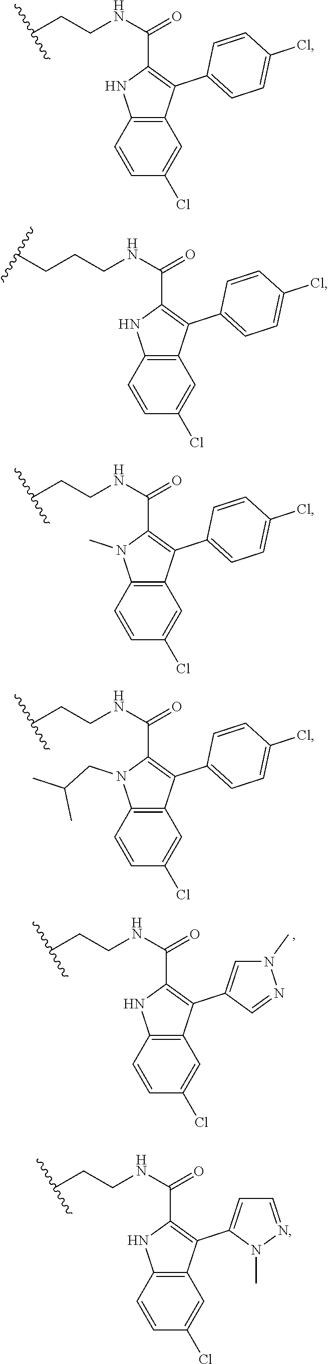

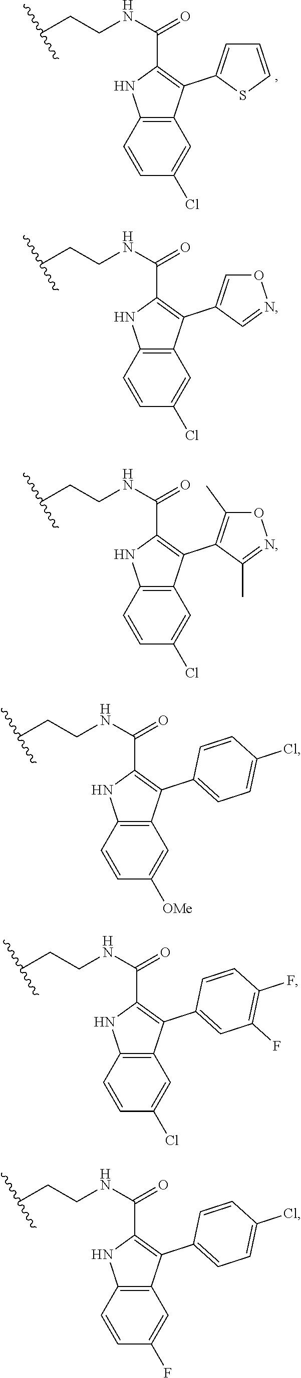

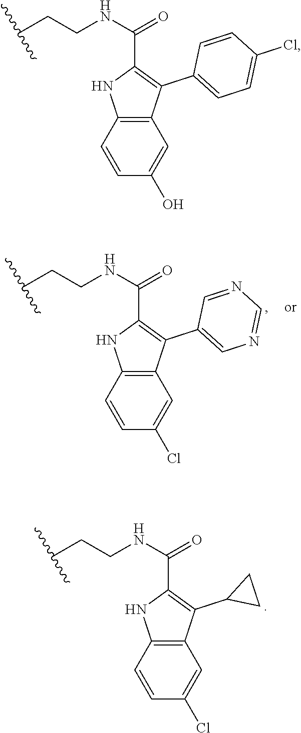 [SMILES: *CCNC(=O)c1[nH]c2ccc(O)cc2c1-c1ccc(Cl)cc1 ; *CCNC(=O)c1[nH]c2ccc(Cl)cc2c1-c1cncnc1 ; *CCNC(=O)c1[nH]c2ccc(Cl)cc2c1C1CC1]

2. A composition comprising the compound of claim 1 and a pharmaceutically-acceptable carrier.

3. A method of treating cancer or neuro-disease in a patient in need thereof, the method comprising administering to the patient a therapeutically effective amount of the compound of claim 1.

4. The method of claim 3, wherein the method comprises treating Ewing sarcoma, prostate cancer, colon cancer, lung cancer, leukemia, lymphoma, rhabdomyosarcoma or rhabdoid tumor.

5. A method for selectively modulating the activity of a target ETS transcription factor in a patient in need thereof, including administering to the patient a therapeutically effective amount of the compound of claim 1.


SEQUENCE LISTING (4 sequences)

SEQ ID NO: 1 - protein, 98 aa, Homo sapiens
PGSGQIQLWQFLLELLSDSANASCITWEGTNGEFKMTDPDEVARRWGERKSKPNMNYDKLSRALRYYYDKNIMTKVHGKRYAYKFDFHGIAQALQPHP

SEQ ID NO: 2 - protein, 98 aa, Homo sapiens
PGSGQIQLWQFLLELLSDSSNSSCITWEGTNGEFKMTDPDEVARRWGERKSKPNMNYDKLSRALRYYYDKNIMTKVHGKRYAYKFDFHGIAQALQPHP

SEQ ID NO: 3 - protein, 452 aa, Homo sapiens
MDGTIKEALSVVSDDQSLFDSAYGAAAHLPKADMTASGSPDYGQPHKINPLPPQQEWINQPVRVNVKREYDHMNGSRESPVDCSVSKCSKLVGGGESNPMNYNSYMDEKNGPPPPNMTTNERRVIVPADPTLWTQEHVRQWLEWAIKEYSLMEIDTSFFQNMDGKELCKMNKEDFLRATTLYNTEVLLSHLSYLRESSLLAYNTTSHTDQSSRLSVKEDPSYDSVRRGAWGNNMNSGLNKSPPLGGAQTISKNTEQRPQPDPYQILGPTSSRLANPGSGQIQLWQFLLELLSDSANASCITWEGTNGEFKMTDPDEVARRWGERKSKPNMNYDKLSRALRYYYDKNIMTKVHGKRYAYKFDFHGIAQALQPHPTESSMYKYPSDISYMPSYHAHQQKVNFVPPHPSSMPVTSSSFFGAASQYWTSPTGGIYPNPNVPRHPNTHVPSHLGSYY

SEQ ID NO: 4 - protein, 460 aa, Homo sapiens
MIQTVPDPAAHIKEALSVVSDEQSLFECAYGTPHLAKTEMTASSSSDYGQTSKMSPRVPQQDWLSQPPARVTIKMECNPSQVNGSRNSPDECSVAKGGKMVGSPDTVGMNYGSYMEEKHMPPPNMTTNERRVIVPADPTLWSTDHVRQWLEWAVKEYGLPDVNILLFQNIDGKELCKMTKDDFQRLTPSYNADILLSHLHYLRETPLPHLTSDDVDKALQNSPRLMHARNTDLPYEPPRRSAWTGHGHPTPQSKAAQPSPSTVPKTEDQRPQLDPYQILGPTSSRLANPGSGQIQLWQFLLELLSDSSNSSCITWEGTNGEFKMTDPDEVARRWGERKSKPNMYDKLSRALRYYYDKNIMTKVHGKRYAYKFDFHGIAQALGPHQQESSLYKYPSDLPYMGSYHAHPQKMNFVAPHPPALPVTSSSFFAAPNPYWNSPTGGYPNTRLPTSHMPSHLGTYY